(19) United States
(12) Patent Application Publication
(10) Pub. No.: US 2026/0335787 A1
(43) Pub. Date: Oct. 29, 2026

(54) Ultrasonic Transducer System for Efficient Hydrocarbon-Based Cannabinoid Extraction from Biomass in Hazardous Environments

(71) Applicant: William Patton, Irvine, CA (US)

(72) Inventor: William Patton, Irvine, CA (US)

(21) Appl. No.: 19/189,901

(22) Filed: Apr. 25, 2025

(51) Int. Cl. B01D 11/02; C07C 37/70; C07D 311/80

(52) U.S. Cl. CPC B01D 11/0265; B01D 11/0207; B01D 11/0219; B01D 11/0288; C07C 37/70; C07D 311/80

(57) ABSTRACT

An ultrasonic transducer system designed for use in solvent-based extraction of cannabinoids from organic biomass within hazardous environments such as C1D1, C1D2, or equivalents. The system comprises a modular mounting assembly configured to attach to extraction columns of various sizes, including but not limited to 4-inch, 6-inch, and 8-inch diameters, using clamp, welded, magnetic, or alternative attachment methods. The system utilizes one or more ultrasonic transducers operating at ultrasonic frequencies to induce cavitation within solvents such as hydrocarbons or ethanol, enhancing compound yield and solvent penetration. It is scalable, corrosion-resistant, and compliant with hazardous location safety standards. An external control unit enables real-time adjustment of frequency and power, with optional integration of machine learning for process optimization. The system is adaptable to batch or continuous-flow extraction and includes thermal management and safety features for reliable operation in volatile environments.

### 1. FIELD OF THE INVENTION

The present invention relates to an ultrasonic transducer system specifically designed for integration with solvent-based extraction columns and vessels. It is intended for use in the extraction of cannabinoids—including but not limited to THCA, CBD, CBG, and THC (Delta-9)—from cannabis, hemp, and other organic biomass materials.

The system is engineered for deployment in hazardous environments such as C1D1, C1D2, or their international equivalents, where flammable or explosive solvents are used. These solvents may include hydrocarbons such as butane and propane, as well as ethanol and other polar or non-polar solvents.

This invention improves upon traditional extraction methods by incorporating ultrasonic cavitation, which significantly enhances solvent penetration and compound release—even at low temperatures (0 °C to −80 °C) commonly used in cannabinoid extraction. The ultrasonic system allows for higher extraction yields, reduced solvent consumption, and shorter processing times, while maintaining compliance with safety standards for volatile environments.

The system features a modular, scalable architecture and supports multiple mounting configurations, including clamp-based, welded, magnetic, and bolted attachments. Transducers can be configured to deliver targeted ultrasonic energy output—up to 2000 watts per column—to suit varying column sizes and extraction volumes. The invention also supports real-time adjustment of ultrasonic frequency and power, enabling it to adapt to different biomass types and extraction goals.

### 2. BACKGROUND AND CHALLENGES IN TRADITIONAL EXTRACTION SYSTEMS

Traditional solvent-based extraction systems used for isolating cannabinoids from biomass materials such as cannabis and hemp often rely on passive soaking, recirculation, or rinsing methods. These systems are typically operated using hydrocarbon solvents (e.g., butane, propane) or ethanol, and must perform under cold extraction conditions to preserve the chemical integrity of cannabinoids and terpenes.

At these low operating temperatures, solvent solubility decreases, reducing extraction efficiency and requiring greater volumes of solvent to achieve complete saturation. This increases overall cycle time, operating costs, and solvent recovery demands. Moreover, the lack of active agitation within the extraction column means the solvent often moves unevenly through the biomass, leaving portions under-extracted and further reducing yield.

Additionally, conventional extraction systems frequently struggle to meet the rigorous safety requirements of hazardous environments such as C1D1, C1D2, or their equivalents. Incomplete containment, unsealed electrical components, or limited safety redundancies increase the risk of exposure to flammable gases and over-pressurization —posing a threat to both operators and facility infrastructure.

The present invention addresses these limitations through the integration of ultrasonic cavitation into the extraction process. Ultrasonic waves create microscopic vapor bubbles within the solvent, which collapse near plant surfaces to disrupt biomass structure and promote deep solvent penetration. This allows for more efficient compound release, even under cold operating conditions, while reducing the volume of solvent required.

The system is further designed with modular adaptability, hazardous-environment safety compliance, and real-time process control, offering a robust and scalable solution that significantly improves extraction performance and operational safety over traditional systems.

### 1. SUMMARY OF THE INVENTION

The present invention provides an ultrasonic transducer system designed for solvent-based extraction of cannabinoids from cannabis, hemp, and other organic biomass. The system integrates with extraction columns and vessels, operating under cold conditions and within hazardous environments such as C1D1, C1D2, or equivalent classifications. It enhances the solvent-to-biomass interaction using ultrasonic cavitation, enabling higher yields, shorter extraction cycles, and reduced solvent use compared to conventional passive or mechanically agitated extraction systems.

This invention is modular, scalable, and solvent-agnostic, with advanced process control and safety features designed for harsh operational environments.

### A. Flexible Mounting Options

The system includes a universal mounting assembly designed to adapt to a wide variety of extraction columns and reactor vessels. It supports multiple securement methods—including modular clamp systems, welded interfaces, magnetic plates, bracketed connections, and bolted flanges.

This flexibility allows manufacturers or processors to integrate the ultrasonic module into both new and existing extraction systems without custom fabrication. It also enables fast serviceability and adaptability across different column sizes (4″, 6″, 8″, and beyond), making the system scalable and versatile for various throughput levels.

### B. Direct Coupling for Enhanced Cavitation Efficiency

The ultrasonic transducer is directly coupled to the wall of the extraction column, which allows ultrasonic energy to be transferred directly into the biomass-solvent mixture without loss through intermediate components.

This direct contact results in precise cavitation targeting—the ultrasonic energy induces microbubble formation and collapse directly adjacent to the biomass. These rapid implosions cause shear forces that break open trichome walls, loosen cellular material, and promote deeper solvent penetration. The result is more complete extraction of cannabinoids and other compounds in less time.

This mechanism avoids the inconsistency of recirculation, soaking, or tumbling methods, where agitation can be uneven and rely on large volumes of solvent.

### C. Effective Solvent Performance at Cold Temperatures

Cannabinoid extractions are often performed at cold or cryogenic temperatures to prevent degradation of volatile compounds like terpenes and to reduce unwanted waxes and chlorophyll in the extract. However, cold temperatures significantly reduce solvent solubility—particularly for hydrocarbons and ethanol.

This invention overcomes that limitation by using ultrasonic cavitation to enhance solvent performance at low temperatures, allowing more efficient diffusion and compound release even when the solvent is less chemically active. This means less solvent is required per extraction, and cooling systems can operate more efficiently since there's no need to elevate temperatures to drive performance.

### D. Scalable Modular Power Configuration

The system features a modular power architecture, allowing ultrasonic transducers to be arranged in one or more locations along the extraction column depending on the size, shape, and target processing volume.

Unlike systems limited to a single transducer or rigid wattage limits, this invention supports scalable power deployment—users may combine multiple transducers in parallel or series to meet custom energy demands. Whether for small 4″ R&D-scale columns or large 8″×48″ industrial vessels, the system provides consistent ultrasonic energy across the entire biomass bed.

This ensures uniform cavitation and avoids over-processing in one area while under-processing in another—a key issue in traditional systems.

### E. Adjustable Ultrasonic Frequency and Power Output

The system allows for real-time control over both frequency and power output. This enables tuning of cavitation strength and depth based on the properties of the biomass being processed:
Lower ultrasonic frequencies penetrate deeper into denser, fibrous plant material (e.g., full-spectrum extractions).
Higher ultrasonic frequencies provide more surface-level agitation for delicate material (e.g., terpene-focused extraction).
Power adjustments allow processors to balance aggressive extraction with compound preservation-ideal when extracting sensitive compounds like THCA or terpenes.

This adaptability enables better optimization per batch, higher quality extracts, and more consistent yields across various biomass types.

### F. Solvent-Agnostic and Ethanol-Compatible Design

While optimized for hydrocarbon solvents like butane and propane, the system is fully compatible with ethanol-based extractions. Ethanol is commonly used in food-grade, pharmaceutical, and GMP-compliant environments due to its polarity and regulatory acceptance.

The system's materials (e.g., stainless steel housing, sealed electrical components, and corrosion-resistant fittings) are engineered to tolerate a wide range of solvent chemistries without degrading performance or safety. This makes the invention applicable across multiple sectors—from small-batch craft processors to industrial ethanol refineries.

### G. Faster Processing and Reduced Solvent Use

By increasing solvent mobility and improving compound release at the cell level, ultrasonic cavitation reduces the total amount of solvent needed per run and accelerates extraction cycles. This improves throughput, reduces energy use in downstream recovery systems, and minimizes solvent losses during processing.

Operators benefit from shorter turnaround time between batches, improved facility efficiency, and lower costs related to solvent storage, handling, and recovery.

### H. Built-in Safety and Thermal Management

Operating in hazardous environments requires high standards for safety and thermal control. This system includes:
A pressure relief valve to prevent over-pressurization during extended or intensive operation
A nitrogen gas cooling line to regulate transducer temperature
A backflow prevention mechanism on the nitrogen inlet to eliminate the risk of reverse pressure or vapor escape
A sealed, corrosion-resistant stainless steel housing with intrinsically safe electrical fittings rated for use in environments with flammable gases

These features ensure long-term durability, fail-safe operation, and compliance with safety standards required in regulated extraction labs and manufacturing facilities.

### I. Optional Machine Learning Integration

An external control module may include a machine learning (ML) system that uses data from sensors monitoring biomass density, solvent concentration, temperature, and extraction time to dynamically adjust ultrasonic parameters. The system can learn from previous runs to refine settings, optimize performance, and maintain consistency even across different biomass profiles or seasonal variations.

This enables data-driven decision making, lowers operator burden, and supports automated batch-to-batch control in high-throughput environments.

### 3. BRIEF DESCRIPTION OF THE DRAWINGS

(Figures to be provided separately)
1. FIG. 1—Top Elevation

FIG. 1 is a top elevation view of the ultrasonic transducer housing assembly, showing the stainless steel spool, sanitary clamps, sealed ultrasonic housing, sealing cover plate, explosion-proof conduit sealing fitting, nitrogen inlet compression fitting with backflow prevention mechanism, and pressure relief valve.
2. FIG. 2—Front Elevation

FIG. 2 is a front elevation view of the ultrasonic transducer housing assembly, illustrating the relative positioning of the stainless steel spool, clamps, sealed housing, cover plate, explosion-proof conduit fitting, nitrogen inlet, and pressure relief valve.
3. FIG. 3—Left Side Elevation

FIG. 3 is a left side elevation view of the ultrasonic housing assembly with stainless steel spool, clamps, sealed ultrasonic housing, and sealing cover plate.
4. FIG. 4—Left Side-Cross Section.

FIG. 4 is a left side cross-sectional view of the ultrasonic housing assembly, showing the internal ultrasonic transducer in relation to the housing and clamps.
5. FIG. 5—Right Side Elevation.

FIG. 5 is a right side elevation view of the ultrasonic housing assembly, showing the sealed housing, cover plate, spool, and conduit fitting.
6. FIG. 6—Right Side-Cross Section.

FIG. 6 is a right side cross-sectional view of the ultrasonic housing assembly, further illustrating the transducer placement and internal arrangement.
7. FIG. 7—3D Perspective.

FIG. 7 is a three-dimensional perspective view of the ultrasonic housing assembly, showing the overall arrangement of the sealed housing, clamps, conduit fitting, and transducer.
8. FIG. 8—3D Wireframe Inside Housing.

FIG. 8 is a three-dimensional wireframe perspective of the ultrasonic housing assembly, illustrating the internal arrangement of the housing, ultrasonic transducer, and fittings.
9. FIG. 9—Exploded Perspective.

FIG. 9 is an exploded perspective view of the ultrasonic transducer housing assembly, showing the relative positions of the sealed housing, clamps, transducer, cover, and fittings prior to assembly.
10. FIG. 10—Column Assembly W/four Ultrasonic Transducer Clamps

FIG. 10 is an elevation view of a column assembly with four ultrasonic transducer clamps mounted to the extraction column.
11. FIG. 11—Nitrogen/Electrical Lines Being Used in a C1D1 Environment

FIG. 11 is a diagrammatic view of nitrogen and electrical line routing in a C1D1 hazardous location, showing the control panels, nitrogen tank, regulator, recovery pump, solvent tank, extraction and distillation columns, and ultrasonic device.

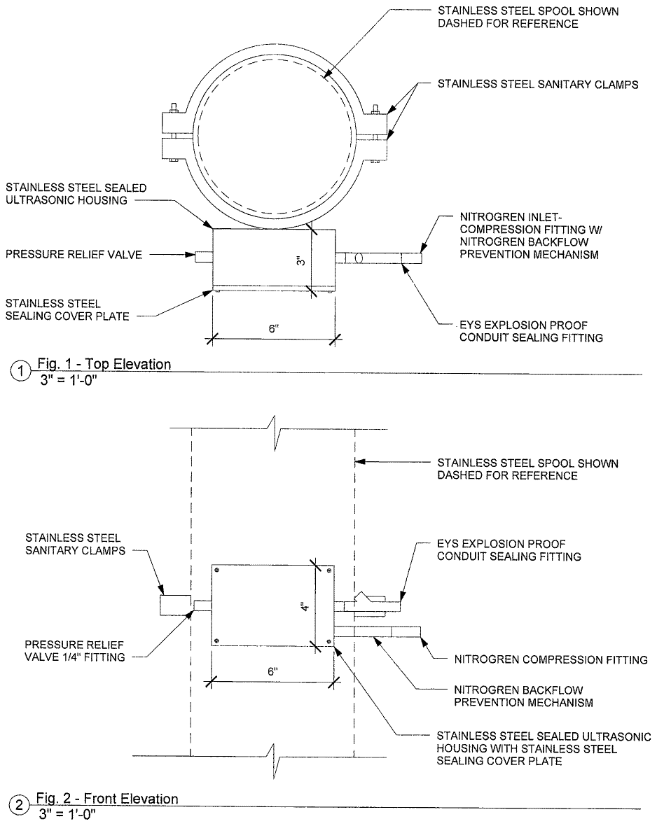

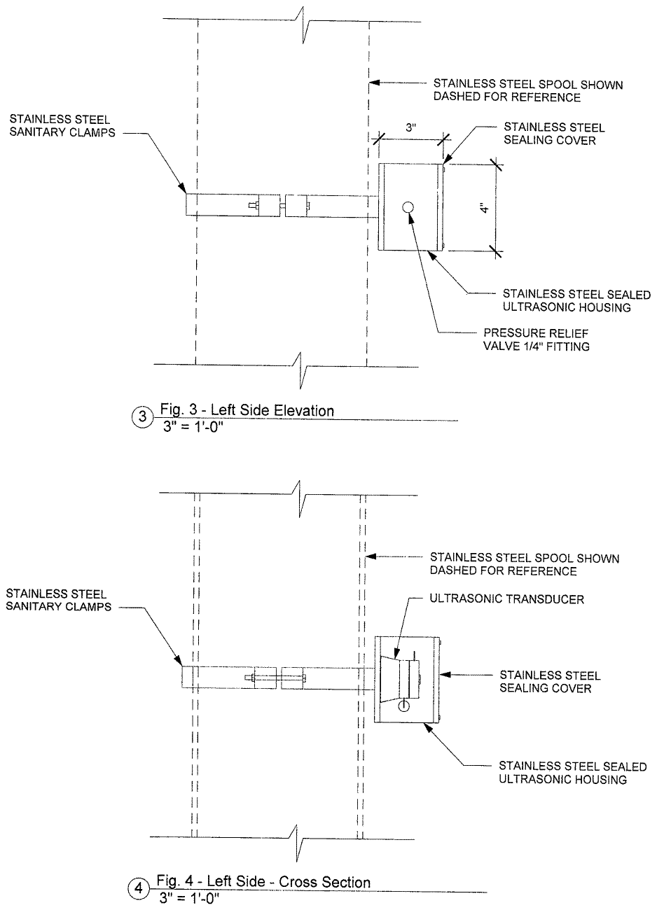

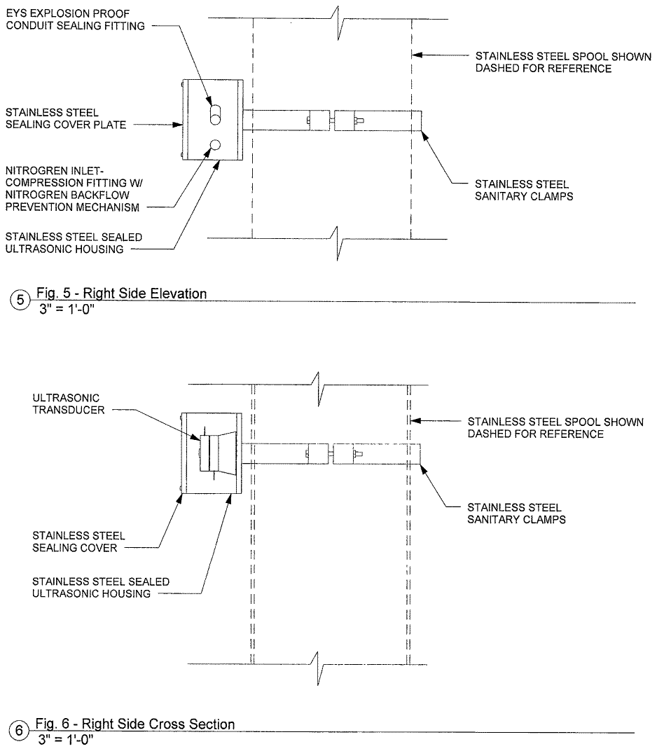

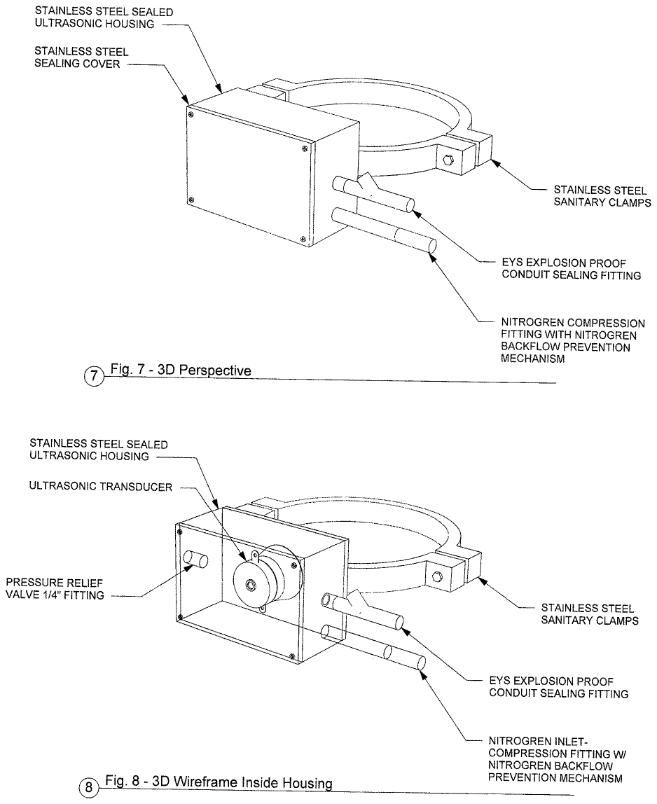

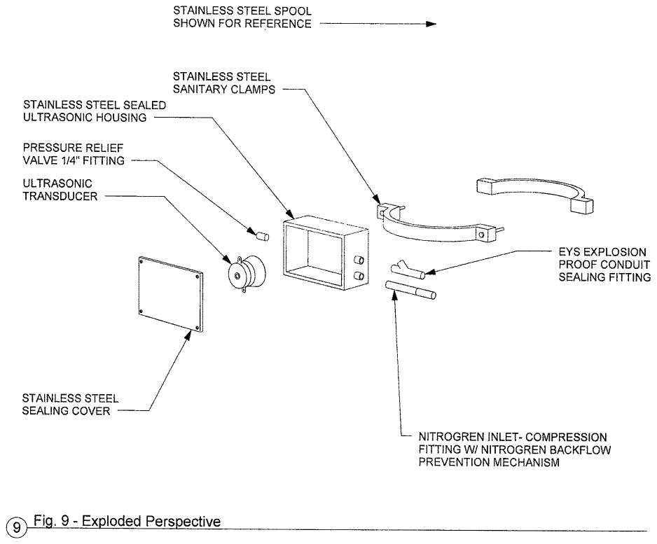

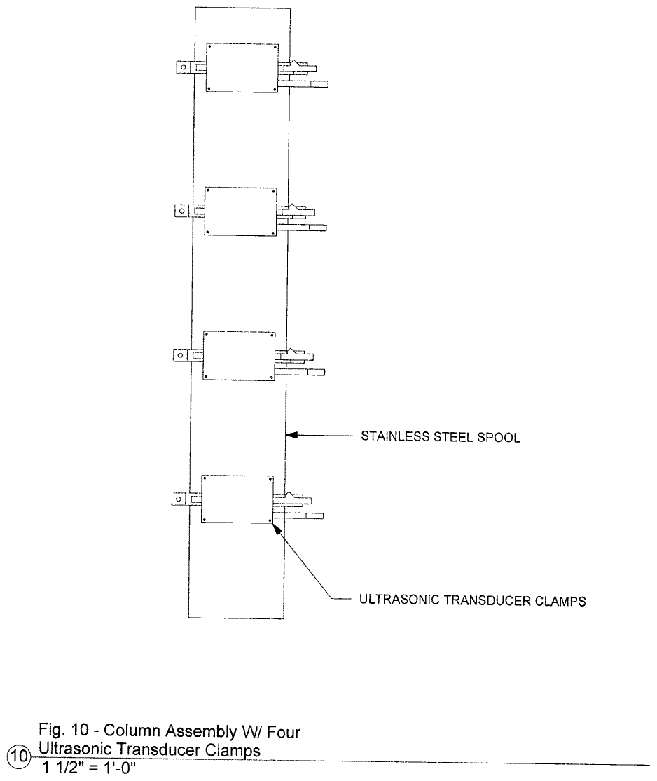

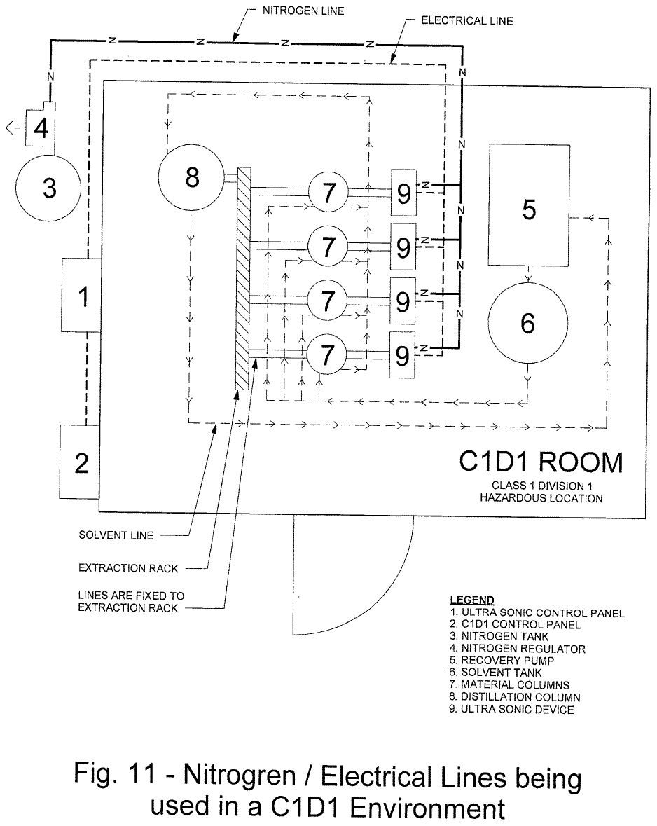

### 4. DETAILED DESCRIPTION OF THE INVENTION

The present invention describes an ultrasonic transducer system developed for integration with solvent-based extraction columns to enhance the extraction of cannabinoids from organic biomass. The system is specifically engineered to operate safely in hazardous environments such as C1D1, C1D2, or international equivalents, and is compatible with hydrocarbon and ethanol-based solvents. Through the use of ultrasonic cavitation, the system improves solvent penetration, increases yield, and significantly reduces processing time.

The invention is constructed around a bolted clamp-style transducer mounting system, providing secure, vibration-resistant attachment to extraction columns of various diameters. It features modular expandability, scalable energy output, and robust safety mechanisms for continuous-duty extraction operations.

### A. Transducer Design and Operation

### Piezoelectric Configuration:

Each ultrasonic transducer employs piezoelectric elements composed of PZT (lead zirconate titanate) ceramics, converting high-frequency electrical signals into mechanical vibrations. These elements are embedded within a hardened steel housing that transmits cavitational energy into the solvent-biased biomass inside the column.

### Energy Uniformity:

Internally, the transducer uses an array of energy-focusing components to deliver even acoustic intensity across the contact area. This design ensures that cavitation occurs uniformly, avoiding areas of under-processing or localized heating.

### Adjustable Ultrasonic Frequencies:

The transducers operate within an ultrasonic frequency range, adjustable based on solvent type, biomass density, and yield goals. Lower frequencies are ideal for penetrating dense material, while higher frequencies produce fine cavitation for surface-level extraction of sensitive compounds.

### B. Bolted Clamp-Style Mounting Assembly

### Mechanical Configuration:

The transducers are mounted directly onto the outer wall of the extraction column using a precision-machined clamp system secured with stainless steel bolts. The clamp is composed of two contoured halves that encase the transducer and compress it firmly against the vessel. A high-conductivity coupling interface is placed between the clamp and the vessel wall to ensure optimal energy transfer.

### Vibration Stability:

The bolted clamp system includes lock washers or vibration-dampening bushings, ensuring long-term securement even during high-power ultrasonic cycles. This assembly resists thermal expansion, mechanical drift, and process-induced vibration.

### Column Compatibility:

The clamp accommodates various standard column diameters (e.g., 4″, 6″, 8″, or larger) and may be reconfigured or resized to fit customized column geometries. The bolted design simplifies servicing and enables quick repositioning or modular expansion of the system along the column body.

### C. Sealed Housing and Environmental Protection

### Corrosion-Resistant Enclosure:

Each transducer is enclosed in a fully sealed stainless steel housing, precision-welded and gasket-reinforced to meet IP67 or better ingress protection standards. The housing prevents moisture, solvent, and vapor ingress into the sensitive transducer components. Chemical and Pressure Shielding:

The seal integrity ensures safe operation even in solvent-heavy, cold, or high-pressure environments. The design complies with safety protocols for operating with flammable chemicals like ethanol, butane, and propane in industrial extraction labs.

### D. Electrical Integration and Safety Compliance

### Explosion-proof Electrical Connections:

All electrical components are routed through C1D1/C1D2-rated conduit fittings, such as explosion-proof bulkhead connectors and grounded cable glands. These prevent spark formation, static discharge, and ingress of flammable gases.

### Overcurrent Protection:

Each electrical line includes thermal cutoff switches, circuit breakers, and fusing to eliminate the risk of overload or thermal runaway.

### Grounding:

The system is electrically grounded through dedicated grounding ports to prevent the buildup of static electricity and ensure safe discharge in volatile working environments.

### E. External Control System

### Manual and Automated Control Interface:

Operators interact with the system via a digital or analog control board that provides real-time access to frequency tuning, power levels, duty cycles, and transducer diagnostics.

### Automation Integration:

The control system can interface with central facility automation (e.g., PLC or SCADA systems) and supports programmable logic sequences for automatic shutdown, feedback-driven tuning, and integration with batch processing schedules.

### Machine Learning (Optional):

A machine learning module may be included to optimize frequency and power settings based on input parameters such as biomass type, solvent type, temperature, and historical yield data.

### F. Nitrogen Cooling and Thermal Control

### Cooling Design:

Each transducer housing features an integrated nitrogen cooling port. Nitrogen gas is routed through a dedicated inlet to cool the housing during extended use or high-energy output conditions.

### Backflow Protection:

The cooling line includes a check valve or one-way control valve to prevent any solvent vapor or internal pressure from escaping into the nitrogen line.

### Fail-Safe Pressure Relief:

An optional venting pathway or PRV (pressure relief valve) ensures safe gas discharge in the event of overpressure buildup within the cooling circuit or housing enclosure.

### G. Modularity and Scalability

### Expandable Configuration:

The bolted clamp system enables users to scale ultrasonic energy delivery by adding multiple transducers along the length of the column. Each clamp assembly may operate independently or in coordinated series through the shared control unit.

### Serviceability:

Transducers may be individually removed for maintenance without interrupting the entire system. This modularity also supports flexibility in processing scale, whether in small-batch operations or full-scale industrial extraction lines.

### Solvent-Agnostic Use:

The materials and construction methods support both non-polar (e.g., butane, propane) and polar (e.g., ethanol) solvents, ensuring chemical compatibility and operational durability across various markets.

### H. Extraction Workflow Overview

1. Transducers are clamped to the column via the bolted mounting system. The ultrasonic transducers are securely attached to the exterior of the extraction column using precision-engineered bolted clamps. The clamps provide firm, vibration-resistant mounting and ensure optimal energy transfer into the vessel wall.
2. Biomass is loaded into an extraction column (e.g., 4″×48″).

Dried and pre-processed cannabis, hemp, or other botanical biomass is packed into a stainless steel extraction column. The material is prepared to ensure even solvent flow and consistent contact with ultrasonic energy zones.
3. Solvent (hydrocarbon or ethanol) is introduced and saturates the biomass.

A chilled solvent such as butane, propane, or ethanol is pumped or gravity-fed into the column. The solvent fully permeates the biomass, initiating the extraction phase.
4. the Control Board Activates Ultrasonic Cavitation at the Desired Power and frequency.

The system's external control unit delivers ultrasonic energy through the transducers, which have been tuned to the optimal frequency and power settings for the biomass type and solvent used.
5. Solvent Rapidly Penetrates Plant Material; Compounds Are Dissolved and released.

Ultrasonic cavitation creates microbubble implosions that disrupt plant cells, rupture trichomes, and accelerate the release of cannabinoids and terpenes into the solvent stream.
6. After Sufficient Time, Solvent Flows to a Distillation Column and Is Recovered back to the solvent tank.

Once extraction is complete, the solvent-extract solution is directed to a distillation or separation system. Solvent is vaporized, condensed, and returned to the storage tank for reuse in future cycles.
7. Nitrogen cooling ensures transducer stability throughout the process.

A continuous flow of nitrogen gas cools the transducer housings via dedicated inlet ports with backflow protection. This prevents overheating during long or high-intensity runs.
8. After solvent has been recovered, extract is collected from the distillation column base.

The purified extract—now free from solvent—is collected from the base of the distillation column. This extract may then be refined, winterized, or formulated depending on end-use requirements.
9. The system resets for the next cycle or transitions into an automated sequence.

Following extraction and recovery, the control system either enters standby or initiates the next programmed run automatically. The operator may adjust parameters or allow machine learning models to optimize future cycles.

### 5. INDUSTRIAL APPLICATIONS

The ultrasonic transducer system described herein is designed to support solvent-based extraction processes, with specific emphasis on the cannabinoid extraction sector, including both hydrocarbon and ethanol-based workflows. The system's innovative use of ultrasonic cavitation, modular bolted clamp mounting, scalable architecture, and real-time process control makes it ideal for deployment in hazardous industrial environments, where safety, performance, and efficiency are paramount.

### A. Cannabis and Hemp Extraction

In the cannabis and hemp industries, there is growing demand for efficient, scalable, and safety-compliant extraction technologies. This ultrasonic system offers significant advantages over traditional methods such as solvent soaking, agitation, and recirculation. By introducing ultrasonic cavitation directly into the extraction column, the system dramatically improves solvent-biomass interaction, reducing saturation time and increasing overall compound release.

Key cannabinoids such as THCA, CBD, CBG, and THC (Delta-9) are efficiently extracted across a range of biomass types. The system's adjustable ultrasonic frequency and power output allows operators to tune cavitation parameters to match plant structure and solvent characteristics in real time, resulting in greater yield consistency, reduced solvent consumption, and shorter extraction cycles.

The bolted clamp mounting system supports easy integration into a wide range of column sizes, from small-batch craft systems to full-scale commercial columns. This makes the system suitable for use in both artisanal operations and high-throughput industrial environments.

### B. Safety in Hazardous Environments

The system is purpose-built to comply with the most stringent safety standards required in hazardous locations such as C1D1, C1D2, and equivalent international classifications. All electrical components are sealed in explosion-proof housings and use intrinsically safe connectors and fail-safe thermal and pressure controls.

A backflow-protected nitrogen cooling system is integrated into each transducer housing to ensure safe and stable operation even during extended ultrasonic cycles. These features collectively mitigate ignition risks, protect operators, and support continuous production workflows in volatile solvent environments.

### C. Ethanol and Food-grade Botanical Extraction

In addition to hydrocarbon workflows, the ultrasonic transducer system is fully compatible with ethanol-based extraction processes. Ethanol's regulatory acceptance and broad solvency profile make it widely used in GMP-certified, pharmaceutical, and food-grade cannabinoid processing operations.

The application of ultrasonic energy in ethanol systems increases surface area exposure of the biomass, promotes deep solvent penetration, and accelerates the release of target compounds such as cannabinoids, flavonoids, terpenes, and other botanical actives.

The system's low-temperature compatibility, combined with its precision-tuned power and frequency control, allows for highly selective extraction in ethanol-based environments where solvent recovery efficiency and extract purity are critical.

### D. Adaptability Across Extraction Sectors

Thanks to its modular, scalable architecture, and solvent-agnostic design, this system is not limited to cannabis or hemp applications alone. It may be applied in:
Botanical nutraceutical extraction
Terpene isolation and refinement
Food ingredient recovery
Pharmaceutical-grade compound processing

With field-replaceable components, universal bolted clamp mounts, and programmable control modules, the system is positioned to support both regulated and industrial-scale extraction operations, offering a long-term, flexible solution for processors aiming to increase efficiency while maintaining compliance.

### E. Adaptability for Global Cannabis Markets

The ultrasonic transducer system is designed to meet the specific needs of the cannabis industry across global markets, ensuring compliance with international regulations and industry standards.
North America: In Canada and the United States, where cannabis extraction is a growing industry, this system's ability to integrate with automated biomass feeding systems and solvent recovery processes provides a cost-effective and efficient solution for both legal medical cannabis and recreational cannabis markets. It is fully compatible with state-regulated systems, ensuring safe extraction practices that meet FDA and EPA standards.
Europe: As cannabis extraction expands within Europe, the system offers a scalable solution for large-scale commercial operations as well as smaller artisanal operations. The ability to meet EU safety standards (e.g., REACH regulations and ISO certifications) makes the system an ideal choice for extraction processes in regions with strict safety and environmental regulations. The system's energy efficiency, solvent recovery capabilities, and reduced environmental impact align with sustainability goals and eco-friendly practices increasingly valued within European markets.

What is claimed is:

1. An ultrasonic transducer system for hydrocarbon extraction columns, comprising: (a) a mounting assembly configured to securely attach to extraction columns of varying sizes, including 4-inch, 6-inch, and 8-inch diameter columns, the mounting assembly comprising a modular two-piece clamp or alternative attachment mechanism including but not limited to welded, magnetic, bolted, or bracket-based connections; (b) a configuration enabling scalability to larger columns through the addition of transducers per mounting position to increase processing capacity; and (c) an ultrasonic cavitation feature that enhances solvent penetration and increases yield during extraction of cannabinoids, including but not limited to THCA, CBD, CBG, and THC (Delta-9), from organic biomass.

2. The system of claim 1, wherein the system is configured for integration with automated biomass feeding systems for real-time adjustment of ultrasonic cavitation parameters based on biomass flow rate and type.

3. The system of claim 1, wherein the system is adaptable for use in a batch-controlled extraction process in which ultrasonic frequency and power output are dynamically adjusted based on biomass volume and solvent quantity.

4. The system of claim 1, wherein the system is scalable to accommodate extraction columns larger than 8 inches in diameter by adding multiple transducers per clamp.

5. The system of claim 4, wherein the additional transducers provide optimized ultrasonic cavitation along the entire length of larger columns, thereby improving yield consistency and reducing processing time.

6. The system of claim 1, wherein the system is environmentally resistant and capable of operating in conditions including high humidity, saltwater exposure, and extreme temperatures without degradation in efficiency.

7. The system of claim 1, wherein the housing and transducer components are constructed from corrosion-resistant materials capable of withstanding exposure to hydrocarbon solvents, including butane, and are suitable for operation in hazardous environments such as C1D1, C1D2, or equivalents.

8. The system of claim 1, further comprising an external control board configured with a machine learning module that automatically adjusts ultrasonic parameters based on real-time sensor data regarding solvent concentration and biomass temperature.

9. The system of claim 8, wherein the machine learning module is configured to analyze historical extraction data to optimize future parameters and predict yield based on biomass characteristics.

10. The system of claim 1, further comprising an emergency shut-off mechanism that automatically deactivates ultrasonic operation upon detection of unsafe pressure, temperature, or gas concentration levels, wherein the system is integrated with a safety-compliant electrical shut-off panel suitable for hazardous environments.

11. The system of claim 1, wherein the system includes intrinsically safe components designed to prevent sparking or ignition during operation in hazardous environments such as C1D1, C1D2, or equivalents.

12. The system of claim 1, wherein the system comprises a pressure relief valve and a nitrogen cooling port to prevent overheating and pressure buildup during extended extraction cycles.

13. The system of claim 1, wherein the ultrasonic housing is IP 67-rated and sealed to protect the transducer from moisture, solvents, and environmental exposure during operation.

14. A method for extracting cannabinoids using the system of claim 1, the method comprising: (a) direct coupling of the ultrasonic transducer to the extraction column to improve energy transfer and cavitation efficiency; and (b) dynamically adjusting ultrasonic frequency and power output based on solvent and biomass conditions to reduce solvent use and shorten extraction time.

15. The method of claim 14, wherein ultrasonic cavitation ruptures trichome walls to increase surface area and accelerate solvent diffusion, enhancing the extraction of cannabinoids, including but not limited to THCA, CBD, CBG, and THC (Delta-9).

16. The system of claim 1, wherein the solvent used for extraction comprises ethanol, either in place of or in combination with hydrocarbon-based solvents.

17. The method of claim 14, wherein the solvent used to extract cannabinoids, including but not limited to THCA, CBD, CBG, and THC (Delta-9), comprises ethanol as the primary extraction solvent.

18. The system of claim 1, wherein the mounting assembly comprises a modular two-piece clamping mechanism designed for tool-free installation and removal from cylindrical extraction columns.

19. The system of claim 1, further comprising a nitrogen cooling line connected to the transducer housing via a pressure-rated fitting, the line including a backflow prevention mechanism configured to prevent solvent vapor or pressure from escaping through the nitrogen inlet during operation in hazardous environments such as C1D1, C1D2, or equivalents.

20. An extraction column or vessel for cannabinoid processing, comprising: a column or vessel body configured to contain biomass and solvent during extraction, the column or vessel body further comprising one or more ultrasonic transducers integrally fabricated, embedded, or built into the wall of the column or vessel and configured to generate ultrasonic cavitation within the solvent during operation in hazardous environments such as C1D1, C1D2, or equivalents.
